(19) United States
(12) Plant Patent Application Publication
(10) Pub. No.: US 2002/0112264 P1
(43) Pub. Date: Aug. 15, 2002

(54) Chrysanthemum named AMOR CORAL

(76) Inventor: Robert Noodelijk, Woubrugge (NL)

Correspondence Address: PARKHURST & WENDEL, SUITE 210, 1421 PRINCE STREET, ALEXANDRIA, VA, 22314, US

(21) Appl. No.: 09/734,605

(22) Filed: Dec. 13, 2000

(51) Int. Cl. A01H 5/00

(52) U.S. Cl. PLT/291

(57) ABSTRACT

A chrysanthemum plant named AMOR CORAL characterized by its medium sized blooms with coral ray florets and prolific branching: natural season flower date September 4-9: blooming for a period of 7 weeks.

RELATED APPLICATIONS

| | | |
|---|---|---|
| Application no. | Filing date | Title |

AMOR
AMOR WHITE

### BACKGROUND OF THE INVENTION

The new plant of the present invention comprises a new and distinct cultivar of Chrysanthemum plant that is a natural occuring sport of a parent chrysanthemum named AMOR. A comparison with Parent chrysanthemum AMOR is also made in this application. The new cultivar was discovered as a whole plant mutation in September 1998 by Rob Noodelijk on a cultivated field in Rijsenhout Holland. The first act of asexual reproduction of AMOR CORAL was accomplished when vegetative cuttings were taken from the initial selection in October 1998 in Rijsenhout Holland.

### BRIEF DESCRIPTION OF THE DRAWINGS

The present invention of a new and distinct variety of chrysanthemum is shown in the accompanying drawings, the color being as nearly true as possible with color photographs of this type.

FIG. 1 shows a plant of the cultivar in full bloom.

FIG. 2 shows the various stages of bloom and foliage of the new cultivar.

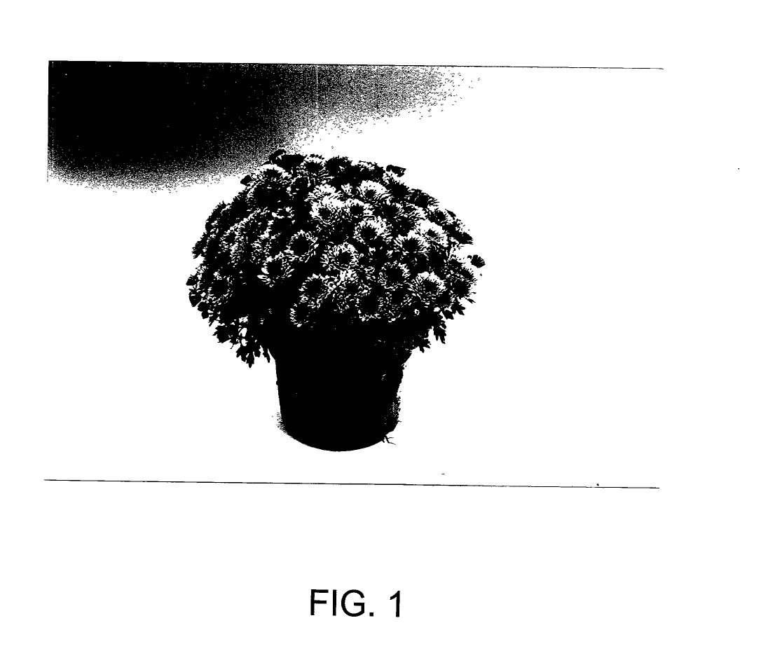

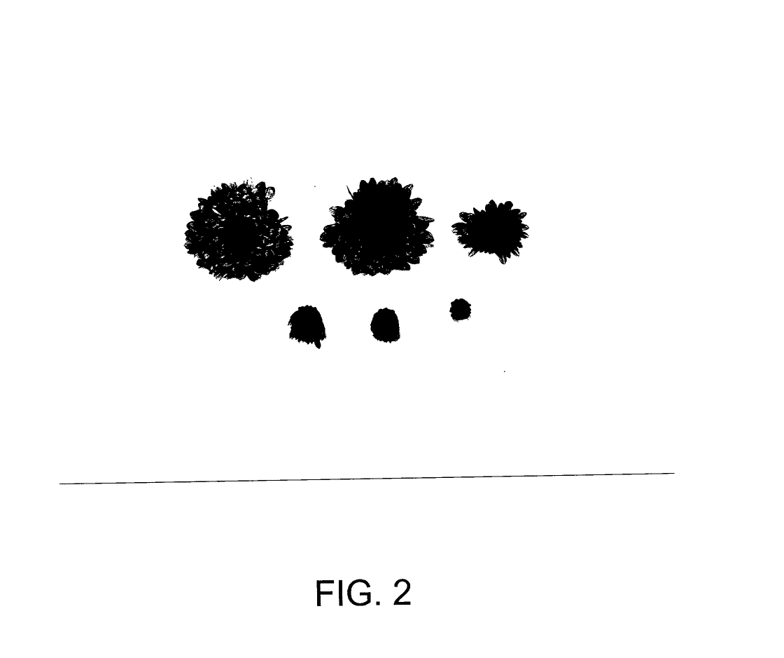

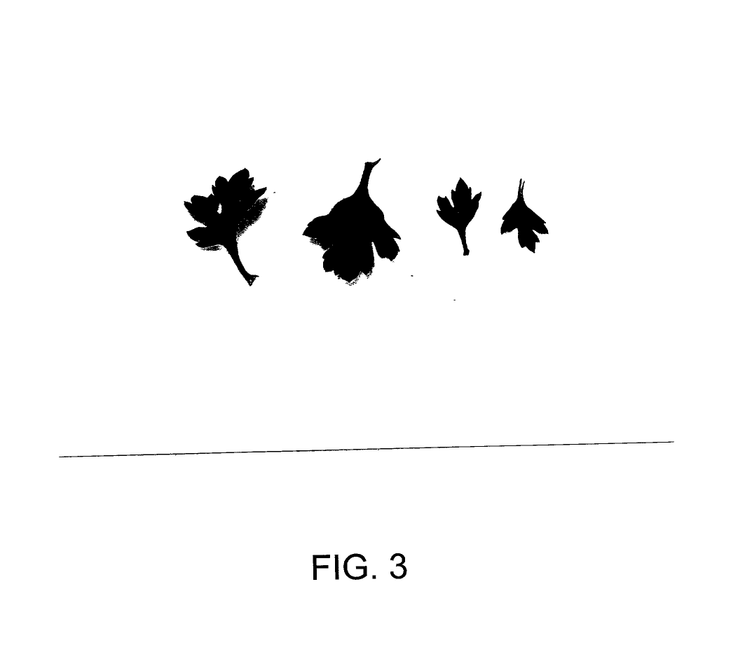

### DESCRIPTION OF THE INVENTION

This new variety of chrysanthemum is of the botanical classification Dendranthema Grandiflora. The observations and measurements were gathered from plants grown out door in Rijsenhout, Holland under natural day length and temperature and planted week 22 in 1999 and 2000. The natural blooming date of this crop was September 4-9 (week 36). The average height of the plants was 30-35 cms. This new variety produces medium sized blooms with coral ray florets and a pink/dark coral center blooming for a period of 7 weeks

When AMOR CORAL and AMOR and AMOR WHITE are being compared the following differences and similarities are noticed: The differences of AMOR CORAL, AMOR and AMOR WHITE are (1) Flower color and (2) Vigour. All other characteristics of AMOR and AMOR CORAL are similar

(1) Flower color; AMOR CORAL is a color mutant of AMOR, bearing coral blooms instead of pink blooms, AMOR WHITE has white blooms instead of pink blooms.

(2) Vigour: The plants of AMOR CORAL tend to be a liffle less vigorous than AMOR. AMOR WHITE is the opposite being a little more vigorous than AMOR. These differences are small.

The following is a description of the plant and characteristics that distinguish AMOR CORAL as a new and distinct variety.

The color designations are taken from the plant itself. Accordingly, any discrepancies between the color designations and the colors depicted in the photographs are due to photographic tolerances.

Botanical Classification

| CULTIVAR | | AMOR | |
|---|---|---|---|
| AMOR CORAL | | WHITE | AMOR |
| Bud | | | |
| Size | Medium, cross- | | |
| | section +/− 1.2 | | |
| | cm height +/− | | |
| | 1.0 cm | | |
| Outside Color | Red-purple | White 155A | Red-purple |
| | 60 D | | 74 C |
| Bloom | | | |
| Size | Medium | | |
| Fully Expanded | 5.0-5.5 | | |
| Borne(number of | Upper and lower | | |
| blooms per | portion plural | | |
| branch) | blooms | | |
| | per branch | | |
| | (approx. 5 blooms | | |
| | per branch) | | |
| Length of | From top till | From top till | From top till |
| lateral branch | bottom 12-13 cm | bottom | bottom |
| | | 14-15 cm | 12-13 cm |
| Lateral branch, | Weak | | |
| Attachment | | | |
| branching | Mounding and | | |
| | prolific | | |
| | with 8-10 breaks | | |
| | after pinching | | |
| Form | Decorative | | |
| Performance (on | 7 weeks | | |
| the plant) | | | |
| Color | | | |
| Center of Flower | Immature red- | Immature | Immature |
| | purple 60 D | White 155A | red-purple |
| | Mature red- | Mature | 74B |
| | purple 60 D | White 155B | Mature |
| | | | red-purple |
| | | | 74D |
| Color of the upper | Red - purple 63 C | White 155D | Red- |
| surface of the | | | purple 74C |
| Majority of the | | | |
| ray-florets | | | |
| Color of the lower | Red - purple 62 C | White 155D | Red- |
| surface of the | | | purple 74D |
| Majority of the | | | |
| ray-florets | | | |
| Tonality from Distance | A mounded | | |
| | decorative garden | | |
| | mum with coral | | |
| | blooms | | |
| Discoloration | To 62 C | None | To red- |
| | | | purple 74D |
| Ray florets | | | |
| Texture | Upper side and | | |
| | under side smooth | | |
| Number | 200-230 | | |
| Cross-section | Concave | | |
| Longitudinal axis of | Incurved to | | |
| the majority | straight | | |
| Shape of tip | Pointed | | |
| Fragrance | Typical | | |
| | chrysanthemum | | |
| Disc Diameter (natural | No disc floret | | |
| season crop when | present | | |
| visible) | | | |
| Reproductive Organs | | | |
| Stamen | Not present | | |
| Pollen | None | | |
| Styles (present in both | Green, thin | | |
| ray and disc florets) | | | |
| Style Length | +/− 4 mm | | |
| Stigmas | Yellow-green | | |
| Stigma Width | +/− 1 mm | | |
| Ovaries | Enclosed in calyx | | |
| Plant | | | |
| Form | A garden mum | | |
| | (pot mum) | | |
| | meant for out | | |
| | door use | | |
| Growth | Medium | | |
| Height (from | 30-35 cms | 32-38 cms | 32-36 cms |
| soil level) | | | |
| Stem Color | Green 138 B | | |
| Stem Strength | Strong | | |
| Stem Brittleness | Present | | |
| Stem Anthocyanin | Present | Absent | Present |
| Coloration | | | |
| Natural blooming date | 4-9 September | | |
| Foliage | | | |
| Color | Upper side green | | |
| | 138 A | | |
| | Under side green | | |
| | 138 B | | |
| Size | small, length | | |
| | +/− 6 cm | | |
| | width +/− | | |
| | 5 cm | | |
| Quantity (number | 12-14 | | |
| per lateral branch) | | | |
| Shape | Oval | | |
| Texture | Fleshy | | |
| Ribs and Veins | Ribs and veins not | | |
| | so well developed | | |
| Edge | Crenated | | |
| Shape of Base of | Acute | | |
| Sinus Between | | | |
| Lateral Lobes | | | |
| Margin of Sinus | Diverging | | |
| Between Lateral | | | |
| Lobes | | | |
| Shape of Base | Asymmetric | | |
| Apex | Mucronate | | |

I claim:

1. A new and distinic variety of chrysanthemum plant as described and illustrated
